(19) United States
(12) Patent Application Publication
(10) Pub. No.: US 2008/0262026 A1
(43) Pub. Date: Oct. 23, 2008

(54) Therapeutic Agents 684

(75) Inventors: Leifeng CHENG, Molndal (SE); Sara Annelie HELGESSON, Molndal (SE)

Correspondence Address: MORGAN LEWIS & BOCKIUS LLP, 1111 PENNSYLVANIA AVENUE NW, WASHINGTON, DC, 20004, US

(73) Assignee: AstraZeneca AB, Sodertalje (SE)

(21) Appl. No.: 12/062,236

(22) Filed: Apr. 3, 2008

Related U.S. Application Data
(60) Provisional application No. 60/909,744, filed on Apr. 3, 2007.

(51) Int. Cl. A61K 31/437; A61P 25/18; A61P 25/28; A61P 25/24

(52) U.S. Cl. 514/300; 546/113

(57) ABSTRACT

Certain 4,5,6,7-tetrahydropyrrolo[3,2-c]pyridin-4-one and 4,5-dihydropyrrolo[3,2-c]pyridin-4-one compounds of formula I, processes for preparing such compounds, their use in the treatment of obesity, psychiatric and neurological disorders, methods for their therapeutic use and pharmaceutical compositions containing them are described.

This application claims the benefit under 35 U.S.C. § 119(e) of Application No. 60/909,744 (US sort 1) filed on 3 Apr. 2007.

### FIELD OF INVENTION

The present invention relates to certain 4,5,6,7-tetrahydropyrrolo[3,2-c]pyridin-4-one and 4,5-dihydropyrrolo[3,2-c]pyridin-4-one compounds of formula I, to processes for preparing such compounds, to their use in the treatment of obesity, psychiatric and neurological disorders, to methods for their therapeutic use and to pharmaceutical compositions containing them.

### BACKGROUND OF THE INVENTION

It is known that certain CB₁ modulators (known as antagonists or inverse agonists) are useful in the treatment of obesity, psychiatric and neurological disorders (WOO 1/70700 and EP 656354).

Co-pending application PCT/GB2006/003695 (WO2007/039740) discloses that compounds of formula A

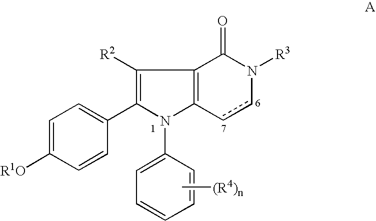

wherein R¹ represents a C₃₋₇alkyl group substituted by one or more fluoro or R¹ represents a C₁₋₇alkylsulphonyl group substituted by one or more fluoro; R² represents cyano, or a C₁₋₄alkyl group optionally substituted by hydroxy or by a group NR^{a}R^{b} in which R^{a} and R^{b} independently represent H or a C₁₋₃alkyl group; R³ represents piperidin-1-yl or cyclohexyl each of which is optionally substituted by one or more groups selected from hydroxy, fluoro or a group NR^{c}R^{d} in which R^{c} and R^{d} independently represent H or a C₁₋₃alkyl group; and
is an optional additional bond between positions 6 and 7;
   R⁴ represents chloro, fluoro, cyano or methyl;
   n is 1, 2 or 3 and each R⁴ is independently selected when n>1;
   and pharmaceutically acceptable salts thereof, are CB₁ modulators
   WO03/027114 discloses the use of 1,5,6,7-tetrahydropyrrolo[3,2-c]pyridine and 1,5,6,7-tetrahydropyrrolo[3,2-c]pyridin-4-one derivatives of formula B

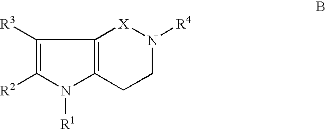

in which
R¹ is phenyl optionally substituted with inter alia one or more halogen;
R² is H, halogen, optionally substituted (C₁-C₉)alkyl, phenyl optionally substituted with one or more halogen, (C₁-C₆)alkyl, (C₂-C₈)alkenyl, (C₂-C₈)alkynyl, (C₁-C₆)alkoxy, (C₁-C₆)alkylthio, trifluoromethyl or trifluoromethoxy;
or a 5- to 10 membered aromatic monocyclic or bicyclic heterocyclic radical which is optionally substituted;
R³ is H, (C₁-C₆)alkyl or benzyl;

### X is —(C═O) or CH₂;

provided that when X is —(C═O) then R⁴ is H; (C₁-C₉)alkyl optionally substituted with one or more hydroxy, benzyloxy, (C₁-C₆)alkoxy, trifluoromethyl, cyano or fluoro; optionally substituted benzyl or phenyl; piperidin-4-yl, piperidin-3-yl or pyrrolidin-3-yl each of which is optionally substituted; or —NR⁵R⁶ in which R⁵ and R⁶ together with the nitrogen atom to which they are attached, form a 5- to 10-membered saturated or unsaturated heterocyclic radical which is optionally substituted;
for the treatment of obesity.

The inventors of WO03/027114 have disclosed the structure activity relationships of this group of compounds in Bioorg and Med Chem Letters 17 (2007) 673-678 in terms of binding affinities at the human CB-1 receptor. In this paper it is disclosed that when R² is phenyl in structure B above then 4-substituents should be kept relatively small, for example 4-F, Cl, CH₃, CH₃O and CF₃, since bulky substituents, for example 4-CH₃S, result in substantial loss of potency.

In contrast we have found that certain bulkier groups in the 4-position have acceptable potency and also appear to have improved physicochemical properties and/or DMPK (Drug Metabolism and Pharmacokinetic) properties and/or pharmacodynamic properties.

### DESCRIPTION OF THE INVENTION

The present invention provides a compound of formula I

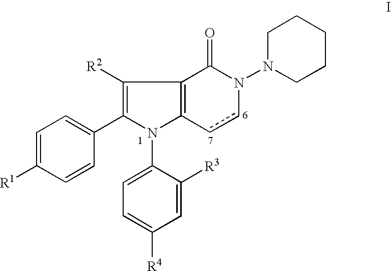

in which
R¹ represents pentyl or pentenyl each of which is optionally substituted by one or more fluoro or R¹ represents a group CH₂NR^{a}R^{b} in which R^{a} and R^{b} together with the nitrogen to which they are attached represent an azetidinyl ring or a pyrrolidinyl ring or a piperidinyl ring each of which is optionally substituted by one or more fluoro;
R² represents cyano or a C₁₋₄alkyl group optionally substituted by hydroxy or a C₁₋₃alkoxy group;
is an optional additional bond between positions 6 and 7;
   R³ represents fluoro or chloro or cyano; and
   R⁴ represents chloro or fluoro or cyano;
   or a pharmaceutically acceptable salt thereof.

In one group of compounds of formula I,

R¹ represents pentyl or pent-1-enyl each of which is substituted by one or more fluoro or
R¹ represents a group CH₂NR^{a}R^{b} in which R^{a} and R^{b} together with the nitrogen to which
they are attached represent an azetidinyl or a pyrrolidinyl ring each of which is substituted by one or more fluoro;
R² represents methyl; —
is an optional additional bond between positions 6 and 7;
   R³ represents chloro; and
   R⁴ represents chloro or fluoro;
   or a pharmaceutically acceptable salt thereof.

In a second group of compounds of formula I,

R¹ represents (3,3-difluoropyrrolidin-1-yl)methyl, (3,3-difluoroazetidin-1-yl)methyl, 5,5,5-trifluoropent-1-en-1-yl, 5,5,5-trifluoropentyl, pent-1-en-1-yl or pentyl;
R² represents methyl; —
is an optional additional bond between positions 6 and 7;
   R³ represents chloro; and
   R⁴ represents chloro or fluoro;
   or a pharmaceutically acceptable salt thereof.

In a particular group of compounds of formula I, R³ and R⁴ each represent chloro.

A particular group of compounds of formula I is represented by formula IA

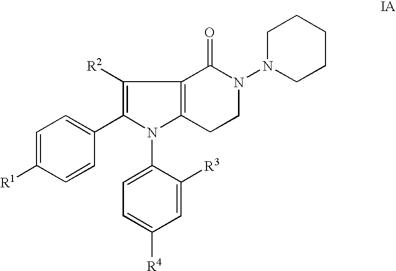

in which
R¹ represents pentyl or pent-1-enyl each of which is substituted by one or more fluoro or
R¹ represents a group CH₂NR^{a}R^{b} in which R^{a} and R^{b} together with the nitrogen to which
they are attached represent an azetidinyl or a pyrrolidinyl ring each of which is substituted by one or more fluoro;
R² represents methyl;
R³ represents chloro; and
R⁴ represents chloro or fluoro;
or a pharmaceutically acceptable salt thereof.

Further values of R¹, R³ and R⁴ in compounds of formula I, and formula IA now follow.

It will be understood that such values may be used where appropriate with any of the definitions, claims or embodiments defined hereinbefore or hereinafter.

In a particular group of compounds of formula IA, R¹ represents (3,3-difluoropyrrolidin-1-yl)methyl, (3,3-difluoroazetidin-1-yl)methyl, 5,5,5-trifluoropent-1-en-1-yl, or 5,5,5-trifluoropentyl. In a particular group of compounds of formula IA, R³ and R⁴ each represent chloro.

Specific compounds of the invention include one or more of the following:
1-(2,4-dichlorophenyl)-2-{4-[(3,3-difluoropyrrolidin-1-yl)methyl]phenyl}-3-methyl-5-piperidin-1-yl-1,5,6,7-tetrahydro-4H-pyrrolo[3,2-c]pyridin-4-one;
1-(2,4-dichlorophenyl)-2-{4-[(3,3-difluoroazetidin-1-yl)methyl]phenyl}-3-methyl-5-piperidin-1-yl-1,5,6,7-tetrahydro-4H-pyrrolo[3,2-c]pyridin-4-one;
1-(2,4-dichlorophenyl)-3-methyl-5-piperidin-1-yl-2-{4-[5,5,5-trifluoropent-1-en-1-yl]phenyl}-1,5,6,7-tetrahydro-4H-pyrrolo[3,2-c]pyridin-4-one;
1-(2,4-dichlorophenyl)-3-methyl-5-piperidin-1-yl-2-[4-(5,5,5-trifluoropentyl)phenyl]-1,5,6,7-tetrahydro-4H-pyrrolo[3,2-c]pyridin-4-one;
1-(2,4-dichlorophenyl)-3-methyl-2-{4-[pent-1-en-1-yl]phenyl}-5-piperidin-1-yl-1,5,6,7-tetrahydro-4H-pyrrolo[3,2-c]pyridin-4-one; and
1-(2,4-dichlorophenyl)-3-methyl-2-(4-pentylphenyl)-5-piperidin-1-yl-1,5,6,7-tetrahydro-4H-pyrrolo[3,2-c]pyridin-4-one;
   or a pharmaceutically acceptable salt thereof.

The compound 1-(2,4-dichlorophenyl)-2-{4-[(3,3-difluoropyrrolidin-1-yl)methyl]phenyl}-3-methyl-5-piperidin-1-yl-1,5,6,7-tetrahydro-4H-pyrrolo[3,2-c]pyridin-4-one or a pharmaceutically acceptable salt thereof.

The compound 1-(2,4-dichlorophenyl)-2-{4-[(3,3-difluoroazetidin-1-yl)methyl]phenyl}-3-methyl-5-piperidin-1-yl-1,5,6,7-tetrahydro-4H-pyrrolo[3,2-c]pyridin-4-one or a pharmaceutically acceptable salt thereof.

The compound 1-(2,4-dichlorophenyl)-3-methyl-5-piperidin-1-yl-2-{4-[5,5,5-trifluoropent-1-en-1-yl]phenyl}-1,5,6,7-tetrahydro-4H-pyrrolo[3,2-c]pyridin-4-one or a pharmaceutically acceptable salt thereof.

The compound 1-(2,4-dichlorophenyl)-3-methyl-5-piperidin-1-yl-2-[4-(5,5,5-trifluoropentyl)phenyl]-1,5,6,7-tetrahydro-4H-pyrrolo[3,2-c]pyridin-4-one or a pharmaceutically acceptable salt thereof.

The compound 1-(2,4-dichlorophenyl)-3-methyl-2-{4-[pent-1-en-1-yl]phenyl}-5-piperidin-1-yl-1,5,6,7-tetrahydro-4H-pyrrolo[3,2-c]pyridin-4-one or a pharmaceutically acceptable salt thereof.

The compound 1-(2,4-dichlorophenyl)-3-methyl-2-(4-pentylphenyl)-5-piperidin-1-yl-1,5,6,7-tetrahydro-4H-pyrrolo[3,2-c]pyridin-4-one or a pharmaceutically acceptable salt thereof

“Pharmaceutically acceptable salt”, where such salts are possible, includes both pharmaceutically acceptable acid and base addition salts. A suitable pharmaceutically acceptable salt of a compound of formula I is, for example, an acid-addition salt of a compound of formula I which is sufficiently basic, for example an acid-addition salt with an inorganic or organic acid for example a hydrochloride salt, a hydrosulphate salt, a sulphate salt, a methanesulphonate salt, a phenylsulphonate salt or a 1,5-naphthalene-disulphonate salt, or for example a salt of a compound of formula I which is sufficiently acidic with a base.

Throughout the specification and the appended claims, a given chemical formula or name shall encompass all stereo and optical isomers and racemates thereof as well as mixtures in different proportions of the separate enantiomers, where such isomers and enantiomers exist, as well as pharmaceutically acceptable salts thereof and solvates thereof such as for instance hydrates. Isomers may be separated using conventional techniques, e.g. chromatography or fractional crystallisation. The enantiomers may be isolated by separation of racemate for example by fractional crystallisation, resolution or HPLC. The diastereomers may be isolated by separation of isomer mixtures for instance by fractional crystallisation, HPLC or flash chromatography. Alternatively the stereoisomers may be made by chiral synthesis from chiral starting materials under conditions which will not cause racemisation or epimerisation, or by derivatisation, with a chiral reagent. All stereoisomers are included within the scope of the invention. All tautomers, where possible, are included within the scope of the invention. The present invention also encompasses compounds containing one or more isotopes for example ¹⁴C, ¹¹C or ¹⁹F and their use as isotopically labelled compounds for pharmacological and metabolic studies.

The present invention also encompasses prodrugs of a compound of formula I that is compounds which are converted into a compound of formula I in vivo.

The following definitions shall apply throughout the specification and the appended claims.

Unless otherwise stated or indicated, the term “alkyl” denotes either a straight or branched alkyl group. Examples of said alkyl include methyl, ethyl, n-propyl, isopropyl, n-butyl, iso-butyl, sec-butyl or t-butyl. Preferred alkyl groups are methyl, ethyl, propyl, isopropyl, butyl and tertiary butyl.

### Methods of Preparation

Compounds of formula I in which R¹, R², R³ and R⁴ are as previously defined may be prepared as shown in General Synthetic Route 1 and in the Examples.

### General Synthetic Route 1

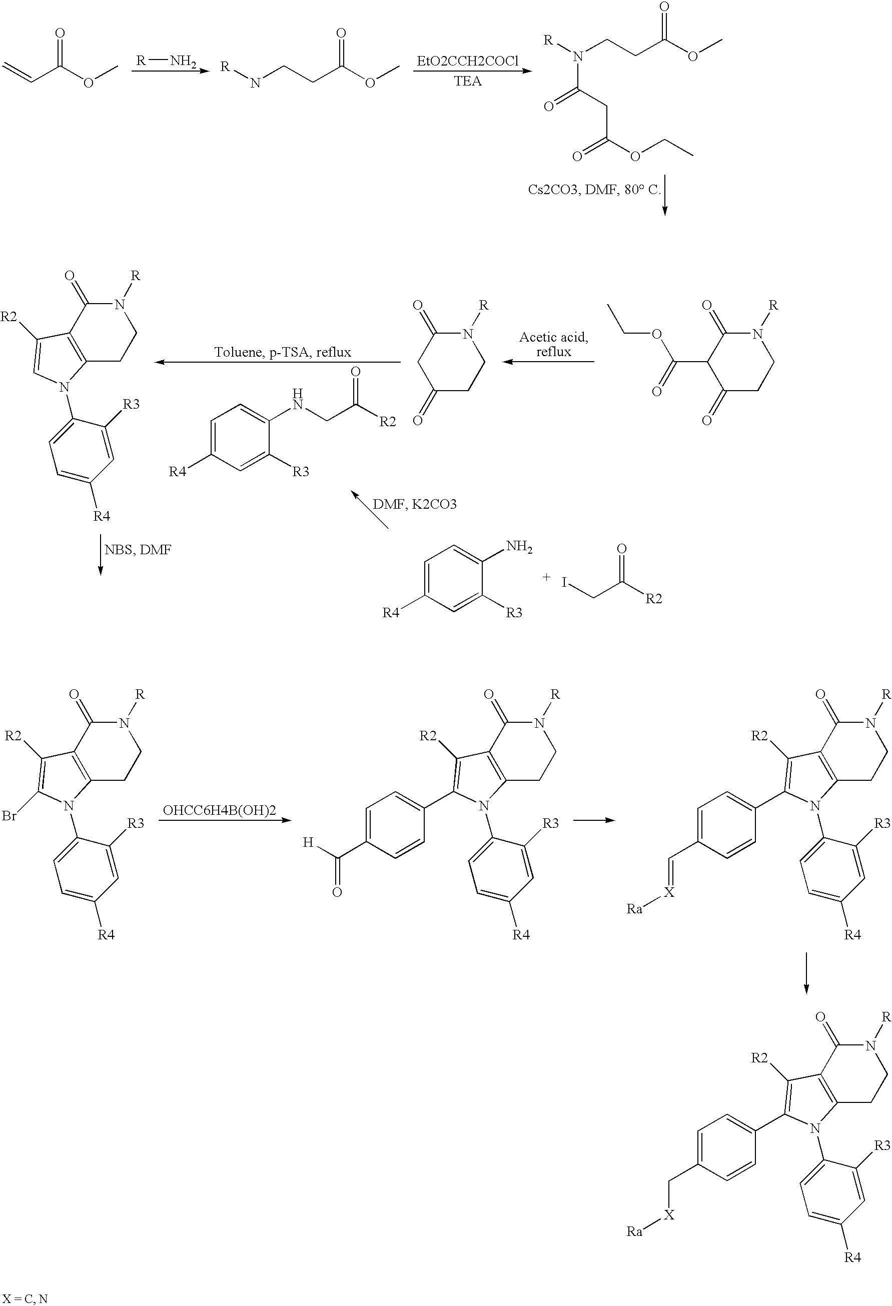

In general synthetic route 1, R is piperidino and R^{s}—X—CH₂— and R^{a}—X═CH— represent groups R¹ in which X is CH₂ or NR_{b}.

Compounds of formula I in which R¹ represents a group CH₂NR^{a}R^{b} in which R^{a} and R^{b} together with the nitrogen to which they are attached represent an azetidinyl ring or a pyrrolidinyl ring or a piperidinyl ring each of which is substituted by one or more fluoro may be prepared by reacting a compound of formula II

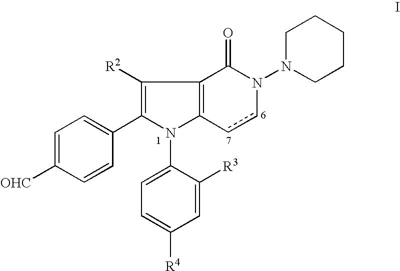

with a compound of formula III

HNR^{a}R^{b} III

in which R^{a} and R^{b} are as defined immediately above in the presence of an inert organic solvent in the presence of a reducing agent at a temperature in the range of 0 to 100° C. Suitable reducing agents include borohydrides for example sodium triacetoxyborohydride. Suitable solvents include dichloroethane. Alternatively a salt of the compound of formula III may be employed in which case a stronger base for example triethylamine is used to regenerate the compound of formula III in situ.

Compounds of formula I in which R¹ represents pentenyl optionally substituted by one or more fluoro may be prepared by reacting a compound of formula II

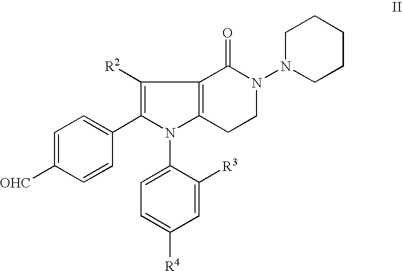

in which R², R³, and R⁴ are as initially defined with a triphenyl(butyl)phosphonium bromide in which the butyl is optionally substituted by one or more fluoro in the presence of a base and an organic solvent at a temperature in the range of 0 to 50° C. Particular bases include sodium hydride, alkyl lithium bases and alkali metal alkoxides. Particular solvents include ethers for example THF.

Compounds of formula I in which R¹ represents pentyl optionally substituted by one or more fluoro may be prepared by reacting a compound of formula I in which R¹ represents pentenyl optionally substituted by one or more fluoro with a reducing agent, for example hydrogen, in the presence of a catalyst, for example Raney nickel, in an inert solvent, for example methanol, optionally in the presence of a base, for example ammonia.

In another aspect the present invention provides an intermediate of formula II

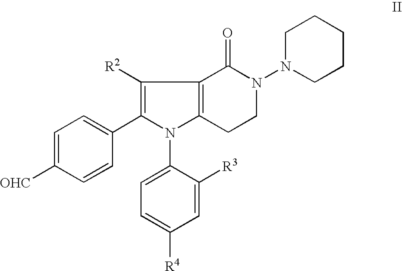

in which R² represents cyano, or a C₁₋₄alkyl group optionally substituted by hydroxy or a C₁₋₃alkoxy group;
R³ represents fluoro or chloro; and
R⁴ represents chloro or fluoro.

Compounds of formula I in which R¹, R², R³, and R⁴ are as initially defined and have a double bond present between positions 6 and 7 may be prepared by reacting a compound of formula I with an oxidising agent for example DDQ or bromine. Alternatively the double bond may be inserted earlier in the reaction sequence by reacting an intermediate 1,5,6,7-tetrahydro-4H-pyrrolo[3,2-c]pyridin-4-one compound with an oxidising agent for example DDQ or bromine.

It will be appreciated by those skilled in the art that during the above processes certain functional groups may require to be protected before a particular reaction or reactions and then de-protected afterwards by methods known to those skilled in the art. Such methods are described in “Protective Groups in Organic Synthesis” by T. W. Greene and P. G. M. Wuts, 3rd Edition John Wiley and Sons, Inc.

### Pharmaceutical Preparations

The compounds of the invention will normally be administered via the oral, parenteral, intravenous, intramuscular, subcutaneous or in other injectable ways, buccal, rectal, vaginal, transdermal and/or nasal route and/or via inhalation, in the form of pharmaceutical preparations comprising the active ingredient or a pharmaceutically acceptable addition salt, in a pharmaceutically acceptable dosage form. Depending upon the disorder and patient to be treated and the route of administration, the compositions may be administered at varying doses.

Suitable daily doses of the compounds of the invention in the therapeutic treatment of humans are about 0.001-10 mg/kg body weight, preferably 0.01-1 mg/kg body weight. Oral formulations are preferred particularly tablets or capsules which may be formulated by methods known to those skilled in the art to provide doses of the active compound in the range of 0.5 mg to 500 mg for example 1 mg, 3 mg, 5 mg, 10 mg, 25 mg, 50 mg, 100 mg and 250 mg.

According to a further aspect of the invention there is also provided a pharmaceutical formulation including any of the compounds of the invention, or pharmaceutically acceptable derivatives thereof, in admixture with pharmaceutically acceptable adjuvants, diluents and/or carriers.

### Pharmacological Properties

The compounds of formula (I) are useful for the treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

The compounds are also potentially useful for the prevention or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

The compounds are also potentially useful for the prevention or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

The compounds are also potentially useful for the treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

The compounds are also potentially useful as agents in treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders. The compounds are also potentially useful as agents in treatment of (esophageal) achalasia.

In another aspect the present invention provides a compound of formula I as previously defined for use as a medicament.

In a further aspect the present invention provides the use of a compound of formula I in the preparation of a medicament for the treatment or prophylaxis of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a further aspect the present invention provides the use of a compound of formula I in the preparation of a medicament for the treatment or prophylaxis of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a further aspect the present invention provides the use of a compound of formula I in the preparation of a medicament for the treatment or prophylaxis of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a further aspect the present invention provides the use of a compound of formula I in the preparation of a medicament for the treatment or prophylaxis of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a further aspect the present invention provides the use of a compound of formula I in the preparation of a medicament for the treatment or prophylaxis of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disoerders (e.g. arthritis deformans, inflammation, inflammatory sequelae of viral encephalitis, osteoarthritis) and orthopedic disorders.

In a still further aspect the present invention provides a method comprising administering a pharmacologically effective amount of a compound of formula I to a patient in need thereof for the prophylaxis or treatment of obesity or being overweight, (e.g., promotion of weight loss and maintenance of weight loss), prevention of weight gain (e.g., medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items), for the treatment of psychiatric disorders such as psychotic and/or mood disorders, schizophrenia and schizo-affective disorder, bipolar disorders, anxiety, anxio-depressive disorders, depression, mania, obsessive-compulsive disorders, impulse control disorders (e.g., Gilles de la Tourette's syndrome), attention disorders like ADD/ADHD, stress, and neurological disorders such as dementia and cognitive and/or memory dysfunction (e.g., amnesia, Alzheimer's disease, Pick's dementia, dementia of ageing, vascular dementia, mild cognitive impairment, age-related cognitive decline, and mild dementia of ageing), neurological and/or neurodegenerative disorders (e.g. Multiple Sclerosis, Raynaud's syndrome, Parkinson's disease, Huntington's chorea and Alzheimer's disease), demyelinisation-related disorders, neuroinflammatory disorders (e.g., Guillain-Barré syndrome).

In a still further aspect the present invention provides a method comprising administering a pharmacologically effective amount of a compound of formula I to a patient in need thereof for the prophylaxis or treatment of dependence and addictive disorders and behaviours (e.g., alcohol and/or drug abuse, pathological gambling, kleptomania), drug withdrawal disorders (e.g., alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances), alcohol and/or drug-induced mood, anxiety and/or sleep disorder with onset during withdrawal, and alcohol and/or drug relapse.

In a still further aspect the present invention provides a method comprising administering a pharmacologically effective amount of a compound of formula I to a patient in need thereof for the prophylaxis or treatment of neurological dysfunctions such as dystonias, dyskinesias, akathisia, tremor and spasticity, treatment of spinal cord injury, neuropathy, migraine, vigilance disorders, sleep disorders (e.g., disturbed sleep architecture, sleep apnea, obstructive sleep apnea, sleep apnea syndrome), pain disorders, cranial trauma.

In a still further aspect the present invention provides a method comprising administering a pharmacologically effective amount of a compound of formula I to a patient in need thereof for the prophylaxis or treatment of immune, cardiovascular disorders (e.g. atherosclerosis, arteriosclerosis, angina pectoris, abnormal heart rhythms, and arrhythmias, congestive heart failure, coronary artery disease, heart disease, hypertension, prevention and treatment of left ventricular hypertrophy, myocardial infarction, transient ischaemic attack, peripheral vascular disease, systemic inflammation of the vasculature, septic shock, stroke, cerebral apoplexy, cerebral infarction, cerebral ischaemia, cerebral thrombosis, cerebral embolism, cerebral hemorrhagia, metabolic disorders (e.g. conditions showing reduced metabolic activity or a decrease in resting energy expenditure as a percentage of total fat-free mass, diabetes mellitus, dyslipidemia, fatty liver, gout, hypercholesterolemia, hyperlipidemia, hypertriglyceridemia, hyperuricacidemia, impaired glucose tolerance, impaired fasting glucose, insulin resistance, insulin resistance syndrome, metabolic syndrome, syndrome X, obesity-hypoventilation syndrome (Pickwickian syndrome), type I diabetes, type II diabetes, low HDL- and/or high LDL-cholesterol levels, low adiponectin levels), reproductive and endocrine disorders (e.g. treatment of hypogonadism in males, treatment of infertility or as contraceptive, menstrual abnormalities/emmeniopathy, polycystic ovarian disease, sexual and reproductive dysfunction in women and men (erectile dysfunction), GH-deficient subjects, hirsutism in females, normal variant short stature) and diseases related to the respiratory (e.g. asthma and chronic obstructive pulmonary disease) and gastrointestinal systems (e.g. dysfunction of gastrointestinal motility or intestinal propulsion, diarrhea, emesis, nausea, gallbladder disease, cholelithiasis, obesity-related gastro-esophageal reflux, ulcers).

In a still further aspect the present invention provides a method comprising administering a pharmacologically effective amount of a compound of formula I to a patient in need thereof for the prophylaxis or treatment of dermatological disorders, cancers (e.g. colon, rectum, prostate, breast, ovary, endometrium, cervix, gallbladder, bile duct), craniopharyngioma, Prader-Willi syndrome, Turner syndrome, Frohlich's syndrome, glaucoma, infectious diseases, urinary tract disorders and inflammatory disorders (e.g. arthritis deformans, inflammation, inflammatory sequelae of viral encephalitis, osteoarthritis) and orthopedic disorders.

The compounds of the present invention are particularly suitable for the treatment of obesity or being overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive), cravings (for drugs, tobacco, alcohol, any appetizing macronutrients or non-essential food items). The compounds of formula (I) are useful for the treatment of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, and neurological disorders such as dementia, neurological disorders (e.g. Multiple Sclerosis), Raynaud's syndrome, Parkinson's disease, Huntington's chorea and Alzheimer's disease. The compounds are also potentially useful for the treatment of immune, cardiovascular, reproductive and endocrine disorders, septic shock and diseases related to the respiratory and gastrointestinal systems (e.g. diarrhea). The compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. The compounds may also eliminate the increase in weight that normally accompanies the cessation of smoking.

In another aspect the present invention provides a compound of formula I as previously defined for use as a medicament.

In a further aspect the present invention provides the use of a compound of formula I in the preparation of a medicament for the treatment or prophylaxis of obesity, psychiatric disorders such as psychotic disorders, schizophrenia, bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms.

In a still further aspect the present invention provides a method of treating obesity, psychiatric disorders such as psychotic disorders such as schizophrenia and bipolar disorders, anxiety, anxio-depressive disorders, depression, cognitive disorders, memory disorders, obsessive-compulsive disorders, anorexia, bulimia, attention disorders like ADHD, epilepsy, and related conditions, neurological disorders such as dementia, neurological disorders (e.g. Multiple Sclerosis), Parkinson's Disease, Huntington's Chorea and Alzheimer's Disease, immune, cardiovascular, reproductive and endocrine disorders, septic shock, diseases related to the respiratory and gastrointestinal systems (e.g. diarrhea), and extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms comprising administering a pharmacologically effective amount of a compound of formula I to a patient in need thereof.

The compounds of the present invention are particulary suitable for the treatment of obesity, e.g. by reduction of appetite and body weight, maintenance of weight reduction and prevention of rebound.

The compounds of the present invention may also be used to prevent or reverse medication-induced weight gain, e.g. weight gain caused by antipsychotic (neuroleptic) treatment(s). The compounds of the present invention may also be used to prevent or reverse weight gain associated with smoking cessation.

The compounds of the present invention are suitable for use in treating the above indications in juvenile or adolescent patient populations.

The compounds of the present invention may also be suitable for use in the regulation of bone mass and bone loss and therefore useful in the treatment of osteoporosis and other bone diseases.

The compounds of the present invention may also be used in the treatment of hepatic diseases, for example hepatic fibrosis, alcoholic liver cirrhosis, chronic viral hepatitis, non-alcoholic fatty liver disease, non-alcoholic steatohepatitis or liver cancer.

### Combination Therapy

The compounds of the invention may be combined with another therapeutic agent that is useful in the treatment of obesity such as other anti-obesity drugs, that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, or G-T motility.

The compounds of the invention may further be combined with another therapeutic agent that is useful in the treatment of disorders associated with obesity such as hypertension, hyperlipidaemias, dyslipidaemias, diabetes, sleep apnea, asthma, heart disorders, atherosclerosis, macro and micro vascular diseases, liver steatosis, cancer, joint disorders, and gallbladder disorders. For example, a compound of the present invention may be used in combination with a another therapeutic agent that lowers blood pressure or that decreases the ratio of LDL:HDL or an agent that causes a decrease in circulating levels of LDL-cholesterol. In patients with diabetes mellitus the compounds of the invention may also be combined with therapeutic agents used to treat complications related to micro-angiopathies.

The compounds of the invention may be used alongside other therapies for the treatment of obesity and its associated complications the metabolic syndrome and type 2 diabetes, these include biguanide drugs, insulin (synthetic insulin analogues) and oral antihyperglycemics (these are divided into prandial glucose regulators and alpha-glucosidase inhibitors).

In another aspect of the invention, the compound of formula I, or a pharmaceutically acceptable salt thereof may be administered in association with a PPAR modulating agent. PPAR modulating agents include but are not limited to a PPAR alpha and/or gamma agonist, or pharmaceutically acceptable salts, solvates, solvates of such salts or prodrugs thereof. Suitable PPAR alpha and/or gamma agonists, pharmaceutically acceptable salts, solvates, solvates of such salts or prodrugs thereof are well known in the art.

In addition the combination of the invention may be used in conjunction with a sulfonylurea. The present invention also includes a compound of the present invention in combination with a cholesterol-lowering agent. The cholesterol-lowering agents referred to in this application include but are not limited to inhibitors of HMG-CoA reductase (3-hydroxy-3-methylglutaryl coenzyme A reductase). Suitably the HMG-CoA reductase inhibitor is a statin.

In the present application, the term “cholesterol-lowering agent” also includes chemical modifications of the HMG-CoA reductase inhibitors, such as esters, prodrugs and metabolites, whether active or inactive.

The present invention also includes a compound of the present invention in combination with an inhibitor of the ileal bile acid transport system (IBAT inhibitor). The present invention also includes a compound of the present invention in combination with a bile acid binding resin.

The present invention also includes a compound of the present invention in combination with a bile acid sequestering agent, for example colestipol or cholestyramine or cholestagel.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration one or more of the following agents selected from:

a CETP (cholesteryl ester transfer protein) inhibitor;
a cholesterol absorption antagonist;
a MTP (microsomal transfer protein) inhibitor;
a nicotinic acid derivative, including slow release and combination products;
a phytosterol compound;
probucol;
an anti-coagulant;
an omega-3 fatty acid;
another anti-obesity compound for example sibutramine, phentermine, orlistat, bupropion, ephedrine, thyroxine;
an antihypertensive compound for example an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic or a vasodilator;
a melanin concentrating hormone (MCH) modulator;
an NPY receptor modulator;
an orexin receptor modulator;
a phosphoinositide-dependent protein kinase (PDK) modulator; or
modulators of nuclear receptors for example LXR, FXR, RXR, GR, ERRα, β, PPARα, β, γ and RORalpha;
a monoamine transmission-modulating agent, for example a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA);
an antipsychotic agent for example olanzapine and clozapine;
a serotonin receptor modulator;
a leptin/leptin receptor modulator;
a ghrelin/ghrelin receptor modulator;
a DPP-IV inhibitor;
or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

According to an additional further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of very low calorie diets (VLCD) or low-calorie diets (LCD).

Therefore in an additional feature of the invention, there is provided a method for the treatment of obesity and its associated complications in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section, or a pharmaceutically acceptable salt, solvate, solvate of such a salt or a prodrug thereof.

Therefore in an additional feature of the invention, there is provided a method of treating hyperlipidemic conditions in a warm-blooded animal, such as man, in need of such treatment which comprises administering to said animal an effective amount of a compound of formula I, or a pharmaceutically acceptable salt thereof in simultaneous, sequential or separate administration with an effective amount of a compound from one of the other classes of compounds described in this combination section or a pharmaceutically acceptable salt, solvate, solvate of such a salt or a prodrug thereof.

According to a further aspect of the invention there is provided a pharmaceutical composition which comprises a compound of formula I, or a pharmaceutically acceptable salt thereof, and a compound from one of the other classes of compounds described in this combination section or a pharmaceutically acceptable salt, solvate, solvate of such a salt or a prodrug thereof, in association with a pharmaceutically acceptable diluent or carrier.

According to a further aspect of the present invention there is provided a kit comprising a compound of formula I, or a pharmaceutically acceptable salt thereof, and a compound from one of the other classes of compounds described in this combination section or a pharmaceutically acceptable salt, solvate, solvate of such a salt or a prodrug thereof.

According to a further aspect of the present invention there is provided a kit comprising:

a) a compound of formula I, or a pharmaceutically acceptable salt thereof, in a first unit dosage form;
b) a compound from one of the other classes of compounds described in this combination section or a pharmaceutically acceptable salt, solvate, solvate of such a salt or a prodrug thereof; in a second unit dosage form; and
c) container means for containing said first and second dosage forms.

According to a further aspect of the present invention there is provided a kit comprising:

a) a compound of formula I, or a pharmaceutically acceptable salt thereof, together with a pharmaceutically acceptable diluent or carrier, in a first unit dosage form;
b) a compound from one of the other classes of compounds described in this combination section or a pharmaceutically acceptable salt, solvate, solvate of such a salt or a prodrug thereof, in a second unit dosage form; and
c) container means for containing said first and second dosage forms.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of obesity and its associated complications in a warm-blooded animal, such as man.

According to another feature of the invention there is provided the use of a compound of the formula I, or a pharmaceutically acceptable salt thereof, and one of the other compounds described in this combination section, or a pharmaceutically acceptable salt, solvate, solvate of such a salt or a prodrug thereof, in the manufacture of a medicament for use in the treatment of hyperlipidaemic conditions in a warm-blooded animal, such as man.

According to a further aspect of the present invention there is provided a combination treatment comprising the administration of an effective amount of a compound of the formula I, or a pharmaceutically acceptable salt thereof, optionally together with a pharmaceutically acceptable diluent or carrier, with the simultaneous, sequential or separate administration of an effective amount of one of the other compounds described in this combination section, or a pharmaceutically acceptable salt, solvate, solvate of such a salt or a prodrug thereof, optionally together with a pharmaceutically acceptable diluent or carrier to a warm-blooded animal, such as man in need of such therapeutic treatment.

Furthermore, a compound of the invention may also be combined with therapeutic agents that are useful in the treatment of disorders or conditions associated with obesity (such as type II diabetes, metabolic syndrome, dyslipidemia, impaired glucose tolerance, hypertension, coronary heart disease, non-alcoholic steatohepatitis, osteoarthritis and some cancers) and psychiatric and neurological conditions.

It will be understood that there are medically accepted definitions of obesity and being overweight. A patient may be identified by, for example, measuring body mass index (BMI), which is calculated by dividing weight in kilograms by height in metres squared, and comparing the result with the definitions.

As the compounds of formula I are useful in causing smoking cessation, preventing weight gain resulting from smoking cessation, treating nicotine withdrawal and preventing nicotine dependence they may also be combined with other compounds known to have one or more of these effects for example nicotine, a nicotine agonist or a partial agonist, a monoamine oxidase inhibitor or antidepressants such as bupropion, doxepine, nortriptyline or an anxiolytic such as buspirone or clonidine.

### Pharmacological Activity

Compounds of the present invention are active against the receptor product of the CB1 gene. The affinity of the compounds of the invention for central cannabinoid receptors is demonstrable in methods described in Devane et al, Molecular Pharmacology, 1988, 34,605 or those described in WO01/70700 or EP 656354.

Alternatively the assay may be performed as follows.

10 μg of membranes prepared from cells stably transfected with the CB1 gene were suspended in 200 μl of 100 mM NaCl, 5 mM MgCl₂, 1 mM EDTA, 50 mM HEPES (pH 7.4), 1 mM DTT, 0.1% BSA and 100 μM GDP. To this was added an EC80 concentration of agonist (CP55940), the required concentration of test compound and 0.1 μCi [³⁵S]-GTPγS. The reaction was allowed to proceed at 30° C. for 45 min. Samples were then transferred on to GF/B filters using a cell harvester and washed with wash buffer (50 mM Tris (pH 7.4), 5 mM MgCl₂, 50 mM NaCl). Filters were then covered with scintilant and counted for the amount of [³⁵S]-GTPγS retained by the filter.

Activity is measured in the absence of all ligands (minimum activity) or in the presence of an EC80 concentration of CP55940 (maximum activity). These activities are set as 0% and 100% activity respectively. At various concentrations of novel ligand, activity is calculated as a percentage of the maximum activity and plotted. The data are fitted using the equation y=A+((B−A)/1+((C/x)ÚD)) and the IC50 value determined as the concentration required to give half maximal inhibition of GTPγS binding under the conditions used.

The compounds of the present invention are active at the CB1 receptor (IC_{50<1} micromolar). Most preferred compounds have IC_{50<200} nanomolar. For example, Example 1 has an IC50 of 5.1 nM. Example 2 has an IC50 of 7.5 nM. Example 3 has an IC50 of 5.0 nM. Example 4 has an IC50 of 2.5 nM. Example 5 has an IC50 of 2.1 nM. Example 6 has an IC50 of 2.1 nM.

The compounds of the invention are believed to be selective CB1 antagonists or inverse agonists. Certain compounds of the invention represent a selection from compounds covered generically by other patent applications. The potency, selectivity profile and side effect propensity may limit the clinical usefulness of hitherto known compounds with alleged CB1 antagonistic/inverse agonistic properties. In this regard, preclinical evaluation of compounds of the present invention in models of gastrointestinal and/or cardiovascular function indicates that they offer significant advantages compared to representative reference CB1 antagonist/inverse agonist agents.

The compounds of the present invention may provide additional benefits in terms of potency, selectivity profile, bioavailability, half-life in plasma, blood brain permeability, plasma protein binding (for example higher free fraction of drug), solubility or in vivo activity compared to representative reference CB1 antagonists/inverse agonist agents.

The compounds of the present invention have improved solubility in organic solvents compared to compounds in the prior art. For example, Example 19 of WO03/027114 (2-(4-chlorophenyl)-1-(2,4-dichlorophenyl)-3-methyl-5-(1-piperidinyl)-1,5,6,7-tetrahydro-4H-pyrrolo-[3,2-c]pyridin-4-one) was found to be insoluble in dimethyl sulfoxide whereas the compounds of the present invention were all soluble in dimethyl sulfoxide. It would be expected that this increase in solubility in organic solvents would lead to improvements in bioavailability and ease of manufacture and formulation.

The utility of the compounds of the present invention in the treatment of obesity and related conditions is demonstrated by a decrease in body weight in cafeteria diet-induced obese mice. Female C57B1/6J mice were given ad libitum access to calorie-dense ‘cafeteria’ diet (soft chocolate/cocoa-type pastry, chocolate, fatty cheese and nougat) and standard lab chow for 8-10 weeks. Compounds to be tested were then administered systemically (iv, ip, sc or po) once daily for a minimum of 5 days, and the body weights of the mice monitored on a daily basis. Simultaneous assessment of adiposity was carried by means of DEXA imaging at baseline and termination of the study. Blood sampling was also carried out to assay changes in obesity-related plasma markers. Compounds of the present invention show superior weight reduction compared to prior art compounds.

### EXAMPLES

### Abbreviations

DCM dichloromethane
DDQ 2,3-dichloro-5,6-dicyano-1,4-benzoquinone
DME dimethoxyethane
DMF dimethylformamide
EtOAc ethyl acetate
NBS N-bromosuccinimide
MeOH methanol
p-TSA 4-toluenesulphonic acid
rt room temperature
TBAF tetrabutylammonium fluoride
TEA triethylamine
TFA trifluoroacetic acid
THF tetrahydrofuran
t triplet
singlet
d doublet
q quartet
qvint quintet
m multiplet
br broad
bs broad singlet
dm doublet of multiplet
bt broad triplet
dd doublet of doublets

### GENERAL EXPERIMENTAL PROCEDURES

Mass spectra were recorded on either a Micromass ZQ single quadrupole or a Micromass LCZ single quadrupole mass spectrometer both equipped with a pneumatically assisted electrospray interface (LC-MS). ¹H NMR measurements were performed on either a Varian Mercury 300 or a Varian Inova 500, operating at ¹H frequencies of 300 and 500 MHz respectively. Chemical shifts are given in ppm with CDCl₃ as internal standard. CDCl₃ is used as the solvent for NMR unless otherwise stated. Purification was performed on a semipreparative HPLC (or hplc—High Performance Liquid Chromatography) with a mass triggered fraction collector, Shimadzu QP 8000 single quadrupole mass spectrometer equipped with 19×100 mm C8 column. The mobile phase used was, if nothing else is stated, acetonitrile and buffer (0.1 M ammonium acetate:acetonitrile 95:5).

### Example 1

### 1-(2,4-dichlorophenyl)-2-{4-[(3,3-difluoropyrrolidin-1-yl)methyl]phenyl}-3-methyl-5-piperidin-1-yl-1,5,6,7-tetrahydro-4H-pyrrolo[3,2-c]pyridin-4-one

### Step 1 3-(Piperidin-1-ylamino)-propionic acid methyl ester

To a solution of 1-aminopiperidine (100 g, 1.00 mol) in dry methanol at 0° C., methyl acrylate (99.0 ml, 1.10 mol) was added dropwise. The resulting mixture was stirred at room temperature overnight. After evaporation of the solvent, heptane was added to the residue, and the white solid (impurity) removed by filtration. The filtrate was concentrated to dryness to afford 80.0 g (43%) of the title compound as a yellow oil.

### Step 2 N-(2-Methoxycarbonyl-ethyl)-N-piperidin-1-yl-maloamic acid ethyl ester

To a solution of 3-(piperidin-1-ylamino)-propionic acid methyl ester (80.0 g, 0.43 mol) in dichloromethane was added triethylamine (71.0 ml, 0.50 mol) followed by slow addition of ethyl malonyl chloride (60.0 ml, 0.47 mol) at 0° C. The resulting slurry was stirred at room temperature for 4 hours. Water was added and the phases separated. The organic phase was dried (Na₂SO₄), filtered and concentrated. Flash chromatography (toluene:EtOAc 9:1-1:1) gave 81.0 g (63%) of the product as an oil which was used without further purification.

### Step 3 2,4-Dioxo-[1,1′]bipiperidinyl-3-carboxylic acid ethyl ester

To a solution of N-(2-methoxycarbonyl-ethyl)-N-piperidin-1-yl-maloamic acid ethyl ester (60.0 g, 0.20 mol) in a mixture of THF (1100 ml) and DMF (490 ml) was added cesium carbonate (195 g, 0.60 mol). The resulting mixture was heated at reflux (80° C.) for 48 hours. The cooled reaction mixture was filtrated and the filtrate evaporated. The combined filtered solid and the filtrate residue were purified by flash chromatography (CH₂Cl₂:MeOH 70:30) to give 15.0 g (28%) of the title compound as a pale yellow oil.

### Step 4 [1,1′]Bipiperidinyl-2,4-dione

The above oil was dissolved in 10% acetic acid (250 ml) and the solution heated at reflux for one hour. The cooled reaction mixture was evaporated, and the residue purified by flash chromatography (CH₂Cl₂:acetone 9:1-1:1) to give 4.00 g (36%) of the title compound as semi-solid.

### Step 5 1-(2,4-Dichloro-phenylamino)-propan-2-one

A mixture of 2,4-dichloroaniline (20.2 g, 0.125 mol), iodoacetone (26.6 g, 0.145 mol) and potassium carbonate (18.1 g, 0.13 mol) in DMF (200 ml) was heated under nitrogen at 100° C. overnight. After cooling to rt, water was added and the mixture extracted with ether (×3). The combined organic extracts were washed with water, dried (Na₂SO₄), filtered and concentrated. Flash chromatography (Heptane:EtOAc 90: 10-80:20) afforded 13.6 g (50%) of the title compound as a brown solid.

### Step 6 1-(2,4-Dichloro-phenyl)-3-methyl-5-piperidin-1-yl-15 6,7-tetrahydro-pyrrolo[3,2-c]pyridine-4-one

To a solution of [1,1′]bipiperidinyl-2,4-dione (520 mg, 2.65 mmol) in dry toluene (25 ml) at room temperature were added 1-(2,4-dichloro-phenylamino)propan-2-one (576 mg, 2.64 mmol) followed by a catalytic amount of p-TSA. The reaction mixture was heated at reflux with a Dean-Stark trap, and 10 ml toluene was collected in the trap. The 1 molar equivalent of p-TSA (250 mg) was added and the reaction mixture heated at reflux for 5.5 hours. After cooling to room temperature, the reaction mixture was evaporated and purified by flash chromatography (heptane:EtOAc gradient) to give 250 mg (25%) of the title compound as a brown solid. A parallel experiment with 1.51 g 1-(2,4-dichloro-phenylamino)-propan-2-one afforded 0.68 g (26%) of the product.

### Step 7 2-Bromo-1-(2,4-Dichloro-phenyl)-3-methyl-5-piperidin-1-yl-1,5,6,7-tetrahydro-pyrrolo[3,2-c]pyridine-4-one

To as solution of 1-(2,4-dichloro-phenyl)-3-methyl-5-piperidin-1-yl-1,5,6,7-tetrahydro-pyrrolo[3,2-c]pyridine-4-one (0.93 g, 2.46 mmol) in DMF (25 ml) was added NBS (0.48, 2.71 mmol) at 0° C. The reaction mixture was stirred at this temperature for one hour and then water was added. The mixture was extracted with ether (×3). The combined ether extracts were dried (Na₂SO₄), filtered and concentrated to give 0.45 g (40%) of the title compound after flash chromatography (heptane:EtOAc gradient).

### Step 8 4-[1-(2,4-dichlorophenyl)-3-methyl-4-oxo-5-piperidin-1-yl-4,5,6,7-tetrahydro-1H-pyrrolo[3,2-c]pyridin-2-yl]benzaldehyde

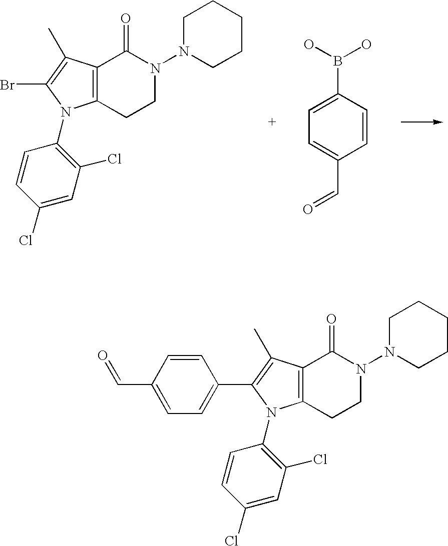

2-Bromo-1-(2,4-dichlorophenyl)-3-methyl-5-piperidin-1-yl-1,5,6,7-tetrahydro-4H-pyrrolo[3,2-c]pyridin-4-one (6.530 g, 14.283 mmol) dissolved in dimethoxyethane (300 ml) was added to (4-formylphenyl)boronic acid (2.356 g, 15.711 mmol) and tetrakis(triphenylphosphine)palladium (2.146 g, 1.857 mmol) under nitrogen followed by the addition of 1M aqueous Na₂CO₃ (90 ml). The reaction mixture was stirred at 60° C. for 24 h. More tetrakis(triphenylphosphine)palladium (2.146 g, 1.857 mmol) and (4-formylphenyl)boronic acid (1.153 g, 7.690 mmol) were added and the reaction continued at 60° C. for another 3 h. The mixture was partitioned between water and ethyl acetate. The organic phase was separated then washed with brine and filtered through celite. The solvent was removed by evaporation and the product purified by flash chromatography (20-30% ethyl acetate in heptane) to yield the product as a slightly yellow solid (4.095 g, 59%). ¹H-NMR (400 MHz, CDCl₃) δ 9.91 (1H, s), 7.71 (2H, d), 7.46 (1H, d), 7.20-7.14 (3H, m), 6.99 (1H, d), 3.75-3.62 (2H, m), 3.62-2.74 (4H, br), 2.74-2.63 (1H, m), 2.63-2.51 (1H, m), 2.40 (3H, s), 1.79-1.50 (4H, m), 1.45-1.35 (2H, m). MS m/z 482 (M+H)⁺.

### Step 9 1-(2,4-dichlorophenyl)-2-{4-[(3,3-difluoropyrrolidin-1-yl)methyl]phenyl}-3-methyl-5-piperidin-1-yl-1,5,6,7-tetrahydro-4H-pyrrolo[3,2-c]pyridin-4-one

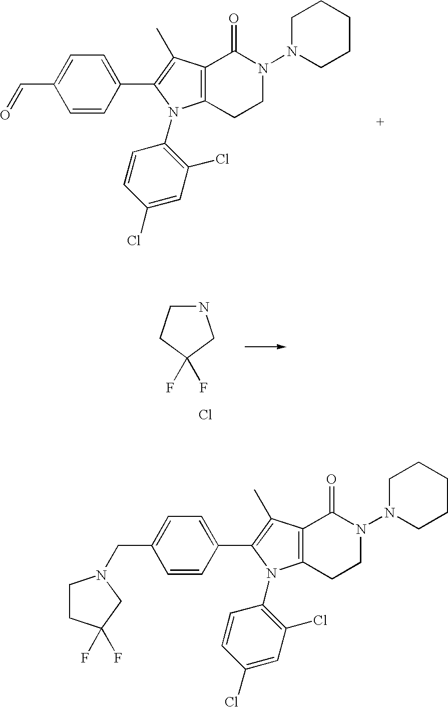

3,3-Difluoropyrrolidine hydrochloride (216 mg, 1.505 mmol) was suspended in dichloroethane (8 ml) and to the suspension was added TEA (168 mg, 1.658 mmol), 4-[1-(2,4-dichlorophenyl)-3-methyl-4-oxo-5-piperidin-1-yl-4,5,6,7-tetrahydro-1H-pyrrolo[3,2-c]pyridin-2-yl]benzaldehyde (400 mg, 0.829 mmol) and sodium triacetoxyborohydride (0.562 mg, 2.653 mmol). The reaction mixture was stirred at rt for 2 h and 15 min. Water (10 ml) was added to the reaction mixture and after 5 min of stirring the phases were separated on a phase separator. The solvent was removed by evaporation and the product purified by hplc to yield the title compound as a white solid after freeze drying (325 mg, 68%). ¹H-NMR (500 MHz, DMSO) δ 7.70 (1H, d), 7.45-7.39 (2H, m), 7.18 (2H, d), 7.02 (2H, d), 3.62 (2H, t), 3.55 (2H, s), 3.14 (4H, m), 2.79 (2H, t), 2.64 (2H, t), 2.61-2.51 (2H, m), 2.26-2.15 (5H, m), 1.60-1.52 (4H, m), 1.40-1.31 (2H, m). HRMS Calcd for [C₃₀H₃₂Cl₂F₂N₄O+H]⁺: 573.200. Found: 573.200.

### Example 2

### 1-(2,4-dichlorophenyl)-2-{4-[(3,3-difluoroazetidin-1-yl)methyl]phenyl}-3-methyl-5-piperidin-1-yl-1,5,6,7-tetrahydro-4H-pyrrolo[3,2-c]pyridin-4-one

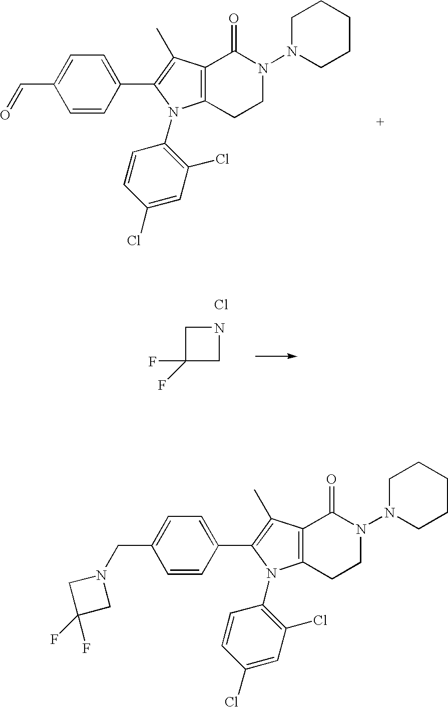

3,3-Difluoroazetidine hydrochloride (172 mg, 1.327 mmol) was suspended in dichloroethane (8 ml) and to the suspension was added TEA (168 mg, 1.658 mmol), 4-[1-(2,4-dichlorophenyl)-3-methyl-4-oxo-5-piperidin-1-yl-4,5,6,7-tetrahydro-1H-pyrrolo[3,2-c]pyridin-2-yl]benzaldehyde (400 mg, 0.829 mmol) and sodium triacetoxyborohydride (562 mg, 2.653 mmol). The reaction mixture was stirred at rt for 3 h. Water (10 ml) was added to the reaction mixture and after 5 min of stirring the phases were separated on a phase separator. The solvent was removed by evaporation and the product purified by hplc to yield the title compound as a white solid after freeze drying (321 mg, 69%). ¹H-NMR (400 MHz, CDCl₃) δ 7.44-7.40 (1H, m), 7.15-7.07 (3H, m), 7.00-6.93 (3H, m), 3.37-3.60 (4H, m), 3.54 (4H, t), 3.47-2.73 (4H, br), 2.71-2.61 (1H, m), 2.59-2.48 (1H, m), 2.33 (3H, s), 1.80-1.51 (4H, m), 1.44-1.33 (2H, m). HRMS Calcd for [C₂₉H₃₀Cl₂F₂N₄O+H]⁺: 559.184. Found: 559.183.

### Example 3

### 1-(2,4-dichlorophenyl)-3-methyl-5-piperidin-1-yl-2-{4-[5,5,5-trifluoropent-1-en-1-yl]phenyl}-1,5,6,7-tetrahydro-4H-pyrrolo[3,2-c]pyridin-4-one

### Step 1 Triphenyl(4,4,4-trifluorobutyl)phosphonium bromide

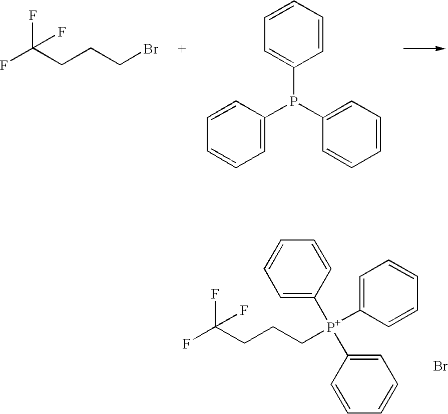

4-Bromo-1,1,1-trifluorobutane (500 mg, 2.618 mmol) and triphenylphosphine (721 mg, 2.749 mmol) were added to dry acetonitrile (2.6 ml) and the mixture was stirred at reflux for 19 h. The solvent was removed by evaporation to give a colourless oil. The oil was dissolved in DCM (3 ml) and added dropwise to a stirred solution of diethyl ether (25 ml) which made the product precipitate. The product was collected by filtration to yield a white solid (994 mg, 84%).

### Step 2 1-(2,4-dichlorophenyl)-3-methyl-5-piperidin-1-yl-2-{4-[5,55-trifluoropent-1-en-1-yl]phenyl}-1,5,6,7-tetrahydro-4H-pyrrolo[3,2-c]pyridin-4-one

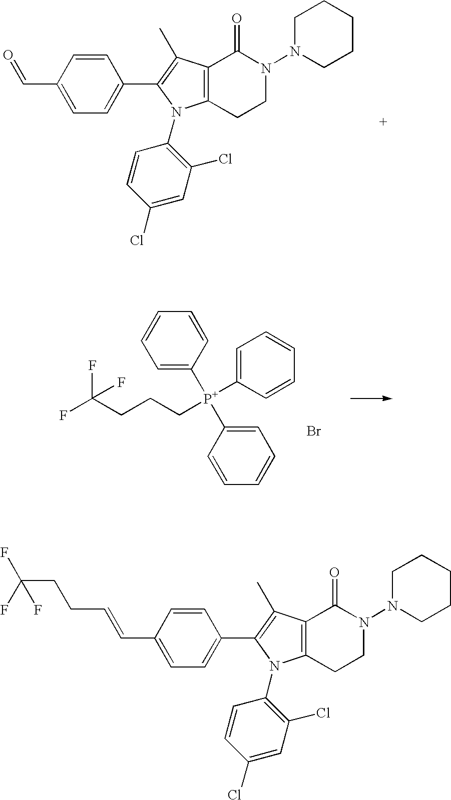

Triphenyl(4,4,4-trifluorobutyl)phosphonium bromide (981 mg, 2.164 mmol) was added to NaH (81 mg, 3.375 mmol) suspended in dry THF (8 ml) under nitrogen. After the mixture had been stirred at rt for 2 h 4-[1-(2,4-dichlorophenyl)-3-methyl-4-oxo-5-piperidin-1-yl-4,5,6,7-tetrahydro-1H-pyrrolo[3,2-c]pyridin-2-yl]benzaldehyde (600 mg, 1.244 mmol) suspended in dry THF (4 ml) was added dropwise. The reaction mixture was stirred at rt overnight and then diethyl ether was added and the mixture filtered through celite. The solvents were removed by evaporation and the product purified by hplc to yield the title compound as a white solid after freeze-drying (395 mg, 55%). ¹H-NMR (400 MHz, CDCl₃) δ 7.42 (1H, d), 7.17-7.10 (1H, m), 7.09-7.03 (2H, m), 7.02-6.93 (3H, m), 6.37 (1H, d), 5.58-5.48 (1H, m), 3.74-3.60 (2H, m), 3.59-2.74 (4H, br), 2.72-2.62 (1H, m), 2.59-2.46 (3H, m), 2.35 (3H, s), 2.23-2.10 (2H, m), 1.82-1.49 (4H, m), 1.43-1.32 (2H, m). HRMS Calcd for [C₃₀H₃₀Cl₂F₃N₃O+H]⁺: 576.180. Found: 576.181.

### Example 4

### 1-(2,4-dichlorophenyl)-3-methyl-5-piperidin-1-yl-2-[4-(5,5,5-trifluoropentyl)phenyl]-1,5,6,7-tetrahydro-4H-pyrrolo[3,2-c]pyridin-4-one

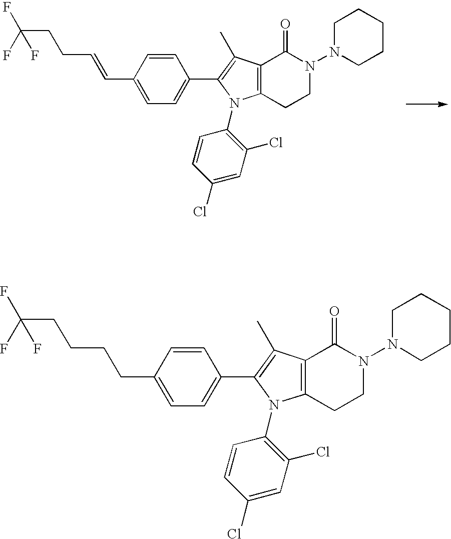

1-(2,4-Dichlorophenyl)-3-methyl-5-piperidin-1-yl-2-{4-[5,5,5-trifluoropent-1-en-1-yl]phenyl}-1,5,6,7-tetrahydro-4H-pyrrolo[3,2-c]pyridin-4-one (140 mg, 0.243 mmol) was dissolved in methanol saturated with NH₃ (4.9 ml). The solution was pumped through a Raney Nickel cartridge in a H-cube at 1 bar, 25° C., flow: 1 ml/min. The solvent was removed by evaporation and the crude product redissolved in methanol saturated with NH₃ (4.9 ml). The reaction was repeated with the same settings. The solvent was removed by evaporation and the product purified by hplc to yield the title compound as a white solid after freeze-drying (95 mg, 68%). ¹H-NMR (400 MHz, CDCl₃) δ 7.43 (1H, d), 7.15-7.10 (1H, m), 7.01-6.90 (5H, m), 3.74-3.60 (2H, m), 3.60-2.73 (4H, br), 2.73-2.62 (1H, m), 2.59-2.49 (3H, m), 2.34 (3H, s), 2.10-1.97 (2H, m), 1.74-1.33 (10H, m). HRMS Calcd for [C₃₀H₃₂Cl₂F₃N₃O+H]⁺: 578.195. Found: 578.194.

### Example 5

### 1-(2,4-dichlorophenyl)-3-methyl-2-{4-[pent-1-en-1-yl]phenyl}-5-piperidin-1-yl-1,5,6,7-tetrahydro-4H-pyrrolo[3,2-c]pyridin-4-one

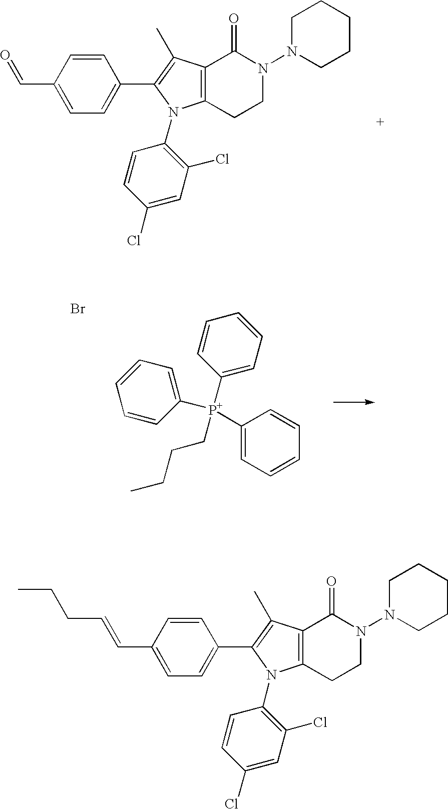

Butyl(triphenyl)phosphonium bromide (993 mg, 2.488 mmol) was added to NaH (60 mg, 2.488 mmol) suspended in dry THF (4 ml) under nitrogen. After the mixture had been stirred at rt for 1 h 4-[1-(2,4-dichlorophenyl)-3-methyl-4-oxo-5-piperidin-1-yl-4,5,6,7-tetrahydro-1H-pyrrolo[3,2-c]pyridin-2-yl]benzaldehyde (400 mg, 0.829 mmol) suspended in dry THF (4 ml) was added dropwise. The reaction mixture was stirred at rt for 4 h, then diethyl ether (5 ml) was added and the mixture filtered through celite. The solvents were removed by evaporation and the product purified by hplc to yield the title compound as a white solid after freeze drying (359 mg, 83%). ¹H-NMR (400 MHz, CDCl₃) δ 7.45-7.42 (1H, m), 7.18-7.09 (3H, m), 7.00-6.94 (3H, m), 6.28 (1H, d), 5.66-5.57 (1H, m), 3.75-3.59 (2H, m), 3.59-2.77 (4H, br), 2.73-2.63 (1H, m), 2.59-2.49 (1H, m), 2.36 (3H, s), 2.30-2.21 (2H, m), 1.79-1.51 (4H, m), 1.49-1.35 (4H, m), 0.91 (3H, t). HRMS Calcd for [C₃₀H₃₃Cl₂N₃O+H]⁺: 522.208. Found: 522.208.

### Example 6

### 1-(2,4-dichlorophenyl)-3-methyl-2-(4-pentylphenyl)-5-piperidin-1-yl-1,5,6,7-tetrahydro-4H-pyrrolo[3,2-c]pyridin-4-one

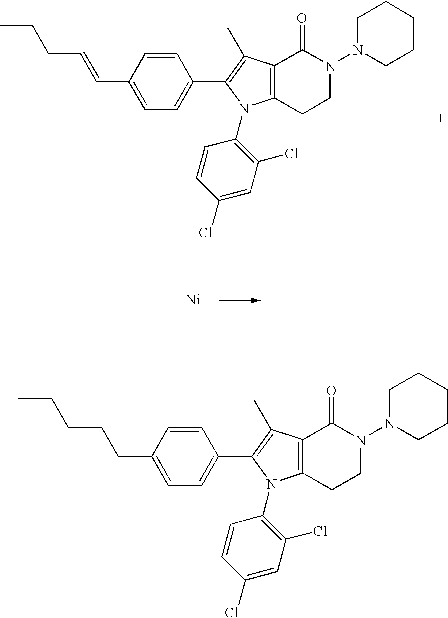

1-(2,4-Dichlorophenyl)-3-methyl-2-{4-[pent-1-en-1-yl]phenyl}-5-piperidin-1-yl-1,5,6,7-tetrahydro-4H-pyrrolo[3,2-c]pyridin-4-one (217 mg, 0.415 mmol) was dissolved in methanol saturated with NH₃ (8.3 ml). The solution was pumped through a Raney Nickel cartridge in a H-cube at 1 bar, 25° C., flow: 1 ml/min. The solvent was removed by evaporation and the product purified by hplc to yield the title compound as a white solid after freeze drying (126 mg, 58%). ¹H-NMR (400 MHz, CDCl₃) δ 7.43 (1H, d), 7.15-7.10 (1H, m), 7.02-6.89 (5H, m), 3.73-3.61 (2H, m), 3.56-2.77 (4H, br), 2.72-2.62 (1H, m), 2.58-2.46 (3H, m), 2.34 (3H, s), 1.77-1.48 (6H, m), 1.46-1.35 (2H, m), 1.34-1.20 (4H, m), 0.85 (3H, t). HRMS Calcd for [C₃₀H₃₅Cl₂N₃₀+H]⁺: 524.224. Found: 524.221.

1: A compound of formula I 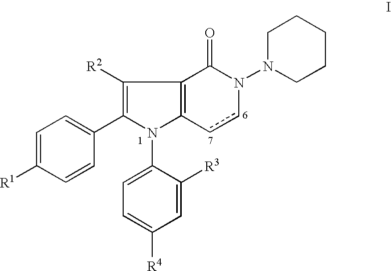
in which
R¹ represents pentyl or pentenyl each of which is optionally substituted by one or more fluoro or R¹ represents a group CH₂NR^{a}R^{b} in which R^{a} and R^{b} together with the nitrogen to which they are attached represent an azetidinyl ring or a pyrrolidinyl ring or a piperidinyl ring each of which is optionally substituted by one or more fluoro,
R² represents cyano, or a C₁₋₄alkyl group optionally substituted by hydroxy or by a C₁₋₃alkoxy group;
is an optional additional bond between positions 6 and 7;
R³ represents fluoro or chloro or cyano; and
R⁴ represents chloro or fluoro or cyano;
or a pharmaceutically acceptable salt thereof.

2. The compound according to claim 1 in which
R¹ represents pentyl or pent-1-enyl each of which is substituted by one or more fluoro or R¹ represents a group CH₂NR^{a}R^{b} in which R^{a} and R^{b} together with the nitrogen to which they are attached represent an azetidinyl or a pyrrolidinyl ring each of which is substituted by one or more fluoro;
R² represents methyl;
is an optional additional bond between positions 6 and 7;
R³ represents chloro; and
R⁴ represents chloro or fluoro;
or a pharmaceutically acceptable salt thereof.

3: A compound according to claim 1
R¹ represents (3,3-difluoropyrrolidin-1-yl)methyl, (3,3-difluoroazetidin-1-yl)methyl, 5,5,5-trifluoropent-1-en-1-yl, 5,5,5-trifluoropentyl, pent-1-en-1-yl or pentyl;
R² represents methyl; —
is an optional additional bond between positions 6 and 7;
R³ represents chloro; and
R⁴ represents chloro or fluoro;
or a pharmaceutically acceptable salt thereof.

4: The compound according to claim 1 in which R³ and R⁴ each represent chloro.

5: The compound according to claim 1 as represented by formula IA 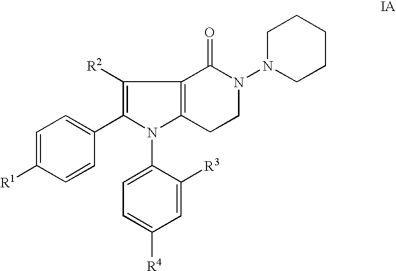
in which
R¹ represents pentyl or pent-1-enyl each of which is substituted by one or more fluoro or R¹ represents a group CH₂NR^{a}R^{b} in which R^{a} and R^{b} together with the nitrogen to which they are attached represent an azetidinyl or a pyrrolidinyl ring each of which is substituted by one or more fluoro;
R² represents methyl;
R³ represents chloro; and
R⁴ represents chloro or fluoro;
or a pharmaceutically acceptable salt thereof.

6: The compound according to claim 5 in which R¹ represents (3,3-difluoropyrrolidin-1-yl)methyl, (3,3-difluoroazetidin-1-yl)methyl, 5,5,5-trifluoropent-1-en-1-yl, or 5,5,5-trifluoropentyl.

7: The compound according to claim 5 in which R³ and R⁴ each represent chloro.

8: The compound according to claim 1 selected from:
1-(2,4-dichlorophenyl)-2-{4-[(3,3-difluoropyrrolidin-1-yl)methyl]phenyl}-3-methyl-5-piperidin-1-yl-1,5,6,7-tetrahydro-4H-pyrrolo[3,2-c]pyridin-4-one;
1-(2,4-dichlorophenyl)-2-{4-[(3,3-difluoroazetidin-1-yl)methyl]phenyl}-3-methyl-5-piperidin-1-yl-1,5,6,7-tetrahydro-4H-pyrrolo[3,2-c]pyridin-4-one;
1-(2,4-dichlorophenyl)-3-methyl-5-piperidin-1-yl-2-{4-[5,55-trifluoropent-1-en-1-yl]phenyl}-1,5,6,7-tetrahydro-4H-pyrrolo[3,2-c]pyridin-4-one;
1-(2,4-dichlorophenyl)-3-methyl-5-piperidin-1-yl-2-[4-(5,5,5-trifluoropentyl)phenyl]-1,5,6,7-tetrahydro-4H-pyrrolo[3,2-c]pyridin-4-one;
1-(2,4-dichlorophenyl)-3-methyl-2-{4-[pent-1-en-1-yl]phenyl}-5-piperidin-1-yl-1,5,6,7-tetrahydro-4H-pyrrolo[3,2-c]pyridin-4-one; and
1-(2,4-dichlorophenyl)-3-methyl-2-(4-pentylphenyl)-5-piperidin-1-yl-1,5,6,7-tetrahydro-4H-pyrrolo[3,2-c]pyridin-4-one;
and pharmaceutically acceptable salts thereof.

9. (canceled)

10: A pharmaceutical formulation comprising a compound of formula I as claimed in any one of claims 1 to 8 and a pharmaceutically acceptable adjuvant, diluent or carrier.

11. (canceled)

12: A method of treating obesity, psychiatric disorders, psychotic disorders, schizophrenia and bipolar disorders, anxiety, anxio-depressive disorders, depression, cognitive disorders, memory disorders, obsessive-compulsive disorders, anorexia, bulimia, attention disorders, epilepsy, and related conditions, neurological disorders, Parkinson's Disease, Huntington's Chorea and Alzheimer's Disease, immune, cardiovascular, reproductive and endocrine disorders, septic shock, diseases related to the respiratory and gastrointestinal system, and extended abuse, addiction and or relapse indications, comprising administering a pharmacologically effective amount of a compound of formula I as claimed in claim 1.

13. (canceled)

14: A process to prepare a compound of formula I as claimed in claim 1 comprising
a) reacting a compound of formula II 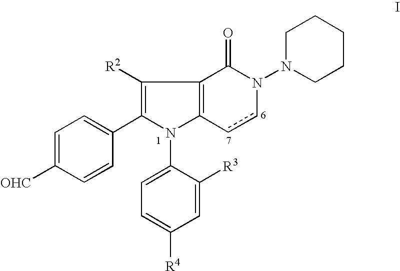
in which R², R³, and R⁴ are as defined in claim 1 with a compound of formula III
HNR^{a}R^{b} III
in which R^{a} and R^{b} together with the nitrogen to which they are attached represent an azetidinyl ring or a pyrrolidinyl ring or a piperidinyl ring each of which is substituted by one or more fluoro in the presence of an inert organic solvent in the presence of a reducing agent at a temperature in the range of 0 to 100° C. to give a compound of formula I in which R¹ represents a group CH₂NR^{a}R^{b} in which R^{a} and R^{b} together with the nitrogen to which they are attached represent an azetidinyl ring or a pyrrolidinyl ring or a piperidinyl ring each of which is substituted by one or more fluoro; or
b) reacting a compound of formula II 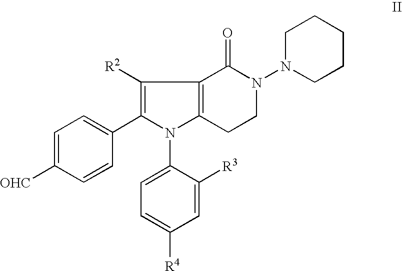
in which R², R³, and R⁴ are as defined in claim 1 with a triphenyl(butyl)phosphonium bromide in which the butyl is optionally substituted by one or more fluoro in the presence of a base and an organic solvent at a temperature in the range of 0 to 50° C. to give a compound of formula I in which R¹ represents pentenyl optionally substituted by one or more fluoro; or
c) reacting a compound of formula I in which R¹ represents pentenyl optionally substituted by one or more fluoro with a reducing agent optionally in the presence of a catalyst in an inert solvent optionally in the presence of a base to give a compound of formula I in which R¹ represents pentyl optionally substituted by one or more fluoro.

15: A compound of formula II 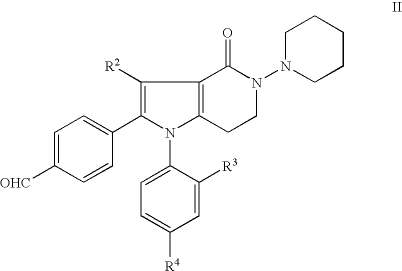
in which
R² represents cyano, or a C₁₋₄alkyl group optionally substituted by hydroxy or by a C₁₋₃alkoxy group;
R³ represents fluoro or chloro or cyano; and
R⁴ represents chloro or fluoro or cyano.
